(19) United States
(12) Patent Application Publication
(10) Pub. No.: US 2005/0044642 A1
(43) Pub. Date: Mar. 3, 2005

(54) Hair colouring composition

(75) Inventor: Gregory Butcher, Amsterdam (NL)

Correspondence Address: NIXON & VANDERHYE, PC, 1100 N GLEBE ROAD, 8TH FLOOR, ARLINGTON, VA, 22201-4714, US

(73) Assignee: Montagne Jeunesse Ltd, Swansea (GB)

(21) Appl. No.: 10/922,960

(22) Filed: Aug. 23, 2004

Related U.S. Application Data
(60) Provisional application No. 60/499,072, filed on Sep. 2, 2003.

(51) Int. Cl. A61K 7/06; A61K 7/13

(52) U.S. Cl. 8/405

(57) ABSTRACT

The invention provides a hair colouring composition in the form of a dispersion comprising (a) from 0.1% to 25% weight, based on the weight of the composition, of a water-dispersible, sulphonate group-containing polyester polymer, (b) from 0.5% to 45% weight, based on the weight of the composition, of a pigment, (c) from 10% to 95% weight, based on the weight of the composition, of water. Also provided, according to further aspects of the invention, are methods for using such a composition and a packaged product containing such a composition.

The present application claims benefit of U.S. Provisional Ser. No. 60/499,072 filed Sep. 2, 2003, the entire contents of which is hereby incorporated by reference.

### FIELD OF THE INVENTION

The present invention relates to a hair colouring composition. More particularly, the present invention relates to a water-based hair colouring composition which incorporates a water-dispersible polymer to impart an even, easily removable coating of colour upon application onto the hair and to methods for using such compositions

### BACKGROUND TO THE INVENTION

Hair colouring compositions are in widespread use within the cosmetic industry and many formulations have been developed and are available to the consumer.

In selecting a product for use, the consumer is faced with a choice between balancing covering ability and water-resistance of the product against its ease of removal. Generally speaking, currently available hair colouring formulations provide either an essentially permanent colouring effect (where the colour is not easily removed by simple washing with shampoo and warm water, for example, but instead ‘grows out’ as the treated hair grows) or they demonstrate little or no water-resistance such that normal hair washing will have the effect of removing the colouring effect. Both types of formulation have their disadvantages. Where the hair has been treated with a ‘permanent’ colouring formulation of the former type, for example, subsequent growth of the hair from the roots leads to a two-tone effect which is clearly evident and generally considered undesirable by the consumer. A disadvantage of the latter, ‘temporary’ type of formulation is that even in gentle rain or spray, some colour will run onto the skin and/or clothes of the user. Furthermore, the colour removal is not uniform and a single wash will not necessarily remove all of the colour from the treated hair.

There therefore remains a continuing need for the development of improved hair colouring formulations and in particular for formulations which provide consistent colour coverage and which are resistant to rain but which nevertheless can readily be removed, as and when required, by conventional washing of the hair with water and shampoo.

The present inventors have surprisingly found that by incorporating into the pigment containing formulation a water-dispersible, sulphonate-group containing, film-forming polymer which forms a continuous film on the hair shaft, it is possible to provide hair colouring products which do not exhibit the disadvantages of the products known in the art.

The use of water-dispersible, film-forming polymers in various cosmetic formulations has previously been described in the literature. Amongst the developments which have been reported are the use of such polymers in cosmetic formulations intended for such applications as mascaras, hair-fixative compositions and as sun-screens.

In U.S. Pat. No. 6,126,948, for example, there are described oil-in-water emulsions containing ionic polymer particles intended for use as topical cosmetic or dermatological preparations, particularly as anti-sun agents. The advantage of these emulsions is stated to be that they have improved fluidity whilst exhibiting good stability, even in the absence of a gelling agent.

Hair cosmetic compositions comprising amine-oxide-containing copolymer in combination with a water-soluble polymer for application as hair sprays, mousses, setting lotions and hair gels are described in U.S. Pat. No. 6,375,932. Hair spray compositions containing a water dispersible polyester or polyesteramide as one component of a multiple polymer system are also described in U.S. Pat. No. 5,158,762. In U.S. Pat. No. 5,314,684 there is described the use of dimethicone polyol to improve the performance of water-based hair spray formulations containing polymeric polyester hair fixatives.

Mascara compositions containing a water dispersible anionic polyester film-forming polymer in combination with microspheres of silica or polymethylmethacrylate (or combinations thereof) are described in U.S. Pat. No. 5,053,221. Mascara compositions containing a hair fixative resin (which may be a film-forming water dispersible polymer) in combination with a curl retention additive are also described in U.S. Pat. No. 5,534,247. It is disclosed that these compositions are preferably in the form of an oil-in-water emulsion.

In order to achieve acceptable mascara compositions, both U.S. Pat. No. 5,053,221 and U.S. Pat. No. 5,534,247 teach that other polymers and/or additional components such as curl retention additives or microspheres are essential. In neither case, is there any suggestion that a water-dispersible film-forming polymer in the absence of such additional components would give an acceptable mascara. Furthermore, the desirable properties in the case of a mascara composition differ significantly from the properties desirable in a hair colouring composition. A mascara has to stiffen, hold and lift the eyebrows whereas a hair colouring composition desirably leaves the hair soft and manageable with the strands of hair remaining separate and smooth to the touch.

In none of the above references is there described the use of water dispersible polymer to deliver pigment to hair in such a way that the colour is readily removable by washing yet is resistant to rain.

### SUMMARY OF THE INVENTION

The present invention provides a hair colouring composition in the form of a dispersion comprising
(a) from 0.1% to 25% weight, based on the weight of the composition, of a water-dispersible, sulphonate group-containing polyester polymer,
(b) from 0.5% to 45% weight, based on the weight of the composition, of a pigment,
(c) from 10% to 95% weight, based on the weight of the composition, of water.

Also provided, according to further aspects of the invention, are methods for using such a composition and a packaged product containing such a composition.

The compositions of the present invention are advantageous as hair colouring compositions. The compositions provide consistency of colour coverage but are substantially water-resistant at ambient temperatures, so that the colour does not wash out on contact with water spray or rain. The compositions of the invention are however readily removable by conventional hair washing processes. By providing hair colouring compositions comprising a water-dispersible sulphonate-group containing polyester polymer, the invention advantageously affords the consumer the possibility of selecting a product which provides an even, rain-resistant coating of colour on the hair, which colouring effect can readily be reversed, as and when desired, by simple hair washing.

This represents a significant advantage to the consumer and meets an unmet commercial need.

### DETAILED DESCRIPTION OF THE INVENTION

As used herein, ‘water-dispersible’ means that the polymer disperses directly in water without the assistance of organic solvents, surfactants or amines.

The water-dispersible, sulphonate-group containing polyester polymer for use according to the invention is a film-forming polymer which forms a hard, clear film that adheres to the hair shaft. Suitably, the polymer has a fast setting speed (for example, of from about 60 to about 300 seconds) and so is fast-drying. The polymer must be substantially resistant to cold water (such as rain) but capable of being removed in the presence of water (preferably warm water) with the physical action of hair washing or in either cold or warm water in the presence of a suitable detergent such as a shampoo.

The presence of ionic substituents on the polyester polymer chain helps to make the dispersion of high levels of pigment possible without the need to form an oil-in-water emulsion to solubilise the polymer (as in the commercially available wash-out hair colouring product ‘Hot Head Streak' n Wash-Out Hair Colour’, Hot Head, Glencoe, Ill. 60022 USA, for example). Compositions according to the invention do not therefore require a high concentration of surfactants to be present which is advantageous as it increases the water resistance of the composition. Any surfactants present are merely to help to improve spreadability and shine of the composition according to the invention and also to solubilise the fragrance in water. Suitably, the level of surfactants in the composition according to the invention is less than 1% by weight of the weight of the composition.

Preferably the polymer for use according to the invention is a polymer of isophthalic or sulphoisophthalic acid, particularly a phthalate/sulphoisophthalate/glycol copolymer.

Particularly preferred polymers for use according to the invention are the diethylene glycol/phthalate/isophthalate/1,4-cyclohexanedimethanol copolymers (INCI name: diglycol/cyclohexanedimethanol/isophthalate/sulphoisophthalate copolymers) which are available from Eastman Chemicals under the name ‘Eastman AQ polymers’.

Eastman AQ polymers are linear, amorphous polyesters whose polymer chain has the general structure HO-G-A-G-A-G-A-G-A-G-A-G-A-G-A-G-A-G-OH where A is a dicarboxylic acid moiety, G is a glycol moiety, —OH are hydroxyl end groups and some of the dicarboxylic acid units have sodiosulpho (SO₃-Na⁺) substituents. The water-dispersibility of these polymers is attributed to the presence of these ionic substituents attached to the polymer chain.

Suitable polyester polymers are commercially available under the product names Eastman AQ 38S, Eastman AQ 48 Ultra and Eastman AQ 55S. These polymers are similar to each other in terms of their physical form, colour and molecular weight and differ mainly in their glass transition temperature (Tg), the number in the product name indicating the dry Tg of each polymer. These polymers may be used individually according to the invention or they can be used in combination.

Polymer AQ 38S is reported to have the typical properties: approximate molecular weight Mn 7000-8000; hydroxyl number, <10; acid number, <2; Tg, ° C. (dry polymer), 35-38; inherent viscosity, dL/g, 0.32-0.40.

Polymer AQ 48 Ultra has the typical properties: approximate molecular weight Mn, 6000-7000; hydroxyl number, <10; acid number, <2; Tg, ° C. (dry polymer), 4548; inherent viscosity, dL/g, 0.26-0.32.

Polymer AQ 55 S has the typical properties: approximate molecular weight Mn 7000-8000; hydroxyl number, <10; acid number, <2; Tg, ° C. (dry polymer), 51-55; inherent viscosity, dL/g, 0.29-0.37.

A particularly preferred polymer for use according to the present invention is Polymer AQ 55 S.

Additional information about the Eastman AQ polymers is contained in the Eastman Chemicals product brochure entitled ‘Eastman AQ Polymers for Cosmetics and Personal Care’, publication no. CB-41, April 2001, the disclosure of which is incorporated herein by reference thereto.

The polyester is present in the composition according to the invention in an amount of from 0.1% to 25% weight, based on the weight of the composition, preferably from 0.5% to 10%, especially from 0.5% to 5% weight, based on the weight of the composition.

In one particular embodiment according to the invention, the overall polymer content of the composition, excluding any silicone polymers present, is no more than 10% by weight of the composition.

The pigment for use according to the invention may be selected from any inorganic or organic pigment conventionally used in hair colouring preparations. It will, of course, be understood that the choice of pigment will depend on the hair colour which it is desired to achieve. The pigment itself may be soluble, partially soluble or insoluble in water provided that when it is incorporated into the overall composition, the pigment is substantially insoluble in water and so therefore is not washed out on contact with rain following application to the hair. Suitably, the pigment is substantially incapable of penetrating the shaft of the hair to facilitate subsequent removal.

Following application of a composition according to the invention, the pigment, together with the polymer to which it is bound, is removed from the hair as and when desired in a removal process involving a single wash with the aid of cold water and the physical action of hair washing or more preferably by washing with warm water and a suitable detergent. Desirably, the pigment used is one which may be removed completely in a single wash but pigments which leave an unnoticeable amount of residue on the hair following a single wash are also acceptable. Typically, the user will not notice residues on the hair at levels of up to 0.1% of the starting level of pigment.

Examples of suitable inorganic pigments which may suitably be used include finely-divided (1 to 300 micron and preferably 10 to 100 micron particle size) iron oxide red, gold or copper or any other variations of iron oxide colours such as ferric ferrocyanide (CI77510), or any other inorganic colour that is not water-soluble in itself. Preferably the inorganic colour is coated on Mica with or without titanium dioxide to give a pearlised effect on hair. The coated inorganic colour is not cold-water soluble. Suitable organic pigments include carmine red (CI 75470), indigoid colours such as Red 122 (CI 73915), or nitroso dyes such toluidine red AKA 221 (CI 12120) and water-insoluble azo dyes.

The pigment is present in the composition according to the invention in an amount of from 0.5% to 45% weight, based on the weight of the composition, preferably from 2% to 20% weight, based on the weight of the composition.

In addition to the polyester polymer and pigment components described above, the hair colour composition according to the invention may suitably also incorporate at least one other component conventionally known as being useful in hair colouring compositions, selected for their known intended use. These additional components, if present, are used in the proportions conventional in hair cosmetic compositions and in an amount effective for their intended purpose. It will, of course, be appreciated that any additional components must not adversely impact on the advantages of the present invention.

Such optional additional components may suitably be selected from the group comprising:—
a) water-miscible organic solvents such as a lower alcohol (for example a C₁-C₅ alcohol, such as ethanol), methylacetone or dimethylether, or other similar rapid-drying solvent to speed up the drying time. The organic solvent, where present, is suitably used in an amount of from 1% to 80% by weight, preferably from 5% to 40% by weight.
b) plasticisers, such as dipropylene glycol, glycerine/propylene glycol and the like, in an amount of from 0.05% to 30% by weight, preferably from 0.25% to 5%;
c) spreading agents or shine-enhancers, such as PEG 75 lanolin or other lanolin-based agents with different PEG chain lengths, polysorbate-type spreading agents and the like, usually in an amount of from 0.05% to 15% by weight, preferably from 0.1% to 5% by weight;
d) de-tangling agents, such as DL panthenol, capable of reducing the ionic charge on hair, in an amount of 0.01% to 5% by weight, preferably from 0.05% to 2.5% by weight. Physical de-tangling agents, such as silicone may suitably be present in an amount of from 0.1% to 25% by weight, preferably from 1% to 10% by weight;
e) suspending agents or thickeners, such as xanthan gum, carbomer and the like, usually in an amount of from 0.05% to 10% by weight, preferably from 0.2% tol % by weight;
f) optical brighteners, conveniently in an amount of from 0.01% to 5% by weight, preferably from 0.05% to 1% by weight;
g) preservatives, such as phenoxyethanol, 2,4, dichlorobenzyl alcohol and the like, conveniently in an amount of from 0.02% to 3.5%, preferably from 0.1% to 1% by weight;
h) fragrances, conveniently in an amount of from 0.1% to 5% by weight, preferably from 0.02% to 1.5% by weight;
i) pearlising and glitter agents, to give extra shine to the colour and sparkle to the hair, usually in an amount of from 0.25% to 10% by weight, preferably from 0.23% to 5% by weight; and
j) pH adjusters, such as triethanolamine or sodium hydroxide and the like, suitably in an amount of from 0.01% to 4% by weight, preferably from 0.05% to 2% by weight.

It will, of course, be understood that the total of all the ingredients in the composition according to the invention is equal to 100% by weight. All percentages are percent by weight of the total composition.

The compositions according to the invention are not suitable as mascara compositions and do not comprise components such as hair curl retention additives and microspheres of silica and/or polymethylmethacrylate which have previously been described as essential components in mascara compositions.

The hair composition according to the invention will desirably have a pH in the range of 5.0-7.5 and preferably 5.5-7.0.

The composition according to the invention may be applied to the hair by a variety of means, for example it may be applied in the form of a cream, gel or lotion. Even distribution of the applied composition may be suitably achieved by using a comb or brush.

According to one aspect, there is provided a product comprising a sachet containing a composition according to the invention and a dispensing device, in fluid communication with the inside of the sachet, by means of which the composition may be dispensed from the sachet.

By ‘sachet’ in this context is meant a container made from flexible or semi-flexible material, typically having a bag-like construction so as to be substantially flat, and which is impermeable to fluids (or at least to the composition according to the invention which it contains) except via the dispensing device. One or more edges of such a container may be gusseted to increase its capacity, as is the case in a pouch. The term ‘sachet’ is therefore intended also to embrace a pouch.

The dispensing device may be any device, typically mechanical in operation, by means of which the composition may be ducted out of the sachet to the hair. It typically comprises one or more fluid ducts through which the composition may exit the sachet. It is preferably manually operable, so that the composition may be forced through by squeezing the sachet. However, it may be more ‘active’ in operation, that is, it may itself act to urge the composition out of the sachet—for instance when the dispensing device is or comprises a fluid pump. In addition, it may comprise fluid flow control means such as a one-way valve, flow-restrictor and/or dosing means by which a pre-determined volume of composition can be dispensed. Dispensing of the composition from the sachet is controlled by dispensing means.

The dispensing device is preferably integral with the sachet so that fluid can only exit the sachet via the dispensing device.

One particularly preferred form of dispensing means incorporates a brush or comb and one or more fluid ducts by means of which the composition may be directed to the region of the comb teeth or brushes so that it may be dispensed during the use of the brush or comb.

FIG. 1 shows a section through a packaged product in accordance with the invention.

There is shown, schematically, a product which comprises a sachet 1 containing a hair colour fluid composition 2. The top edges of the sachet have been welded to a dispensing device generally labelled 3, which comprises a shoulder or “boat” portion 4, a brush or comb 5 and a fluid duct 6 which leads from inside the sachet, through the shoulder portion and into the comb region. The fluid composition 2 may be dispensed to the comb, via the duct 6, by squeezing the sachet 1. The user may thus apply the composition 2 whilst combing his or her hair.

In a product of the type shown in FIG. 1, parts corresponding to the shoulder portion 4 and/or the fluid duct 6 may be provided separately to another external component of the dispensing device, such as a comb or brush or a pump fitting, which may be for instance snap-fitted, screwed, welded or otherwise secured to the shoulder portion or fluid duct at an appropriate time during manufacture. The fluid duct 6 may be defined in either or both of the internal and external components of the device.

A suitable material from which to make the sachet 1 is a laminate comprising (i) a 12 μm outer layer of polyester optionally with graphics printed (eg, by a gravure reverse printing process) on its inside face; (ii) a 12 μm middle layer of aluminium foil; and (iii) a 75 μm inner layer of low density polyethylene (LDPE).

The outer polyester layer provides a highly durable anti-rub protection. The foil middle layer provides additional rigidity to the sachet, particularly since it has excellent “dead-fold” properties, and its metallic gloss gives an attractive and high quality appearance. A 12 μm aluminium layer provides virtually a 100% barrier to all gases with a water vapour transmission rate (WVTR) of significantly better than 0.3 g/m² per 24 hours at 38° C. and 90% RH.

The LDPE inner layer provides a barrier to outside gases and excellent thermal sealing.

It has the additional benefit over normal linear low density polyethylene (LLDPE) of permitting complex shape die-cutting, which in turn permits the ‘radiusing’ of the sachet corners to reduce the risk of scratching or cutting of the user's skin on sharp corners

The hair colouring composition of the present invention may be prepared by dispersing the constituent components in water in known manner, conventional in the art. Conveniently, an aqueous dispersion is prepared from the sulphonate-group containing polyester directly and the pigment and any additional components are added to the aqueous dispersion of the polyester.

The present invention is illustrated by the following non-limiting examples. All percentages are given by weight of the composition unless otherwise indicated.

### EXAMPLE

A hair colouring composition according to the invention was prepared by blending together the following ingredients according to the general method given below.

Formulation for red hair colour:

| Trade name | Inci Name | % w/w | Supplier |
|---|---|---|---|
| Water (1) | Aqua | 33.31 | |
| MP Diol | Methylpropanediol, | 0.4 | Honeywill & |
| | Methylpentanediol | | Stein |
| Solan E | PEG 75 Lanolin | 0.25 | S. Black |
| AQ55S | Diglycol/CHDM/Isophtha- | 2.0 | Eastman |
| | lates/SIP copolymer | | Chemicals |
| DL Panthenol | Panthenol | 0.1 | Surfachem |
| Keltrol RD | Xanthan Gum | 0.55 | S. Black |
| Ultrez 10 (4%) | Carbomer | 6.0 | Surfachem¹ |
| Soln. | | | |
| Water (2) | Aqua | 20.0 | |
| TEA 99% BP | Triethanolamine | 0.20 | BASF |
| Tinopal-CBS-X | Disodium distyrylbiphenyl | 0.15 | Ciba |
| | disolfonate, sodium chloride | | |
| Phenoxyethanol | Phenoxyethanol | 0.6 | Nipa |
| (A) | | | |
| Dichlorobenzyl | Dichlorobenzyl alcohol | 0.15 | BASF |
| Alcohol | | | |
| Fragrance-Fresh | Parfum | 0.08 | Independent |
| Trees | | | |
| Fragrances | | | |
| Polysorbate 20 | Polysorbate 20 | 0.16 | Croda/ |
| (D) | | | Chemicals |
| Tocopherol | Tocopherol | 0.05 | Unichem |
| Ethanol-DB96 | Alcohol Denat. | 20 | Alcohols Ltd |
| Silicone | Decamethylcyclopenta- | 5.0 | S. Black |
| DC5225C | siloxane, | | |
| | PEG/PPG-18/18 dimethicone | | |
| Timica Extra | CI77019 (Mica), CI77891 | 1.0 | Engelhard |
| Bright 1500 | (Titanium dioxide) | | |
| Timica Silver | CI77019 (Mica), CI77891 | 1.0 | Engelhard |
| Sparkle 5500 | (Titanium dioxide). | | |
| Pigment (Pearl- | CI77019 (Mica), CI77491 | 9.0 | Eckart |
| escent): Prestige | (Iron oxide-red), CI77891 | | |
| Fire Red | (Titanium dioxide). | | |

¹The level of solid polymer is 0.24% w/w in the final formulation.

Method of Manufacture

1) Into a side vessel, weigh the water (1) and MP Diol.

2) Add the PEG 75 Lanolin and start heating. When the temperature reaches 60° C., add the AQ55S and then raise and maintain the temperature at 80-85° C. for 25-30 minutes to ensure that the AQ55S is totally dissolved.

3) Add the Keltrol RD and panthenol and wait for 20 minutes while stirring. Start the cooling process.

4) Into the main mixing vessel, add the water (2) and create a vortex. Add the Ultrez 10 and mix until it is fully dispersed and no lumps are present. Pass it through a re-circulating pump fitted with an in-line filter fitted with 200400 micron mesh filter, to ensure that there are no lumps.

5) Neutralise the Ultrez 10 using TEA 50% solution to pH 6-7.

6) When the temperature reaches about 60° C. set up a pump with a filter of about 400 micron on the end of the outlet of the pump and transfer the premix in the side vessel at 60° C. and below into the Ultrez 10 phase while stirring vigorously for 20 minutes and then start re-circulating the bulk from bottom to the top while stirring vigorously and cooling the bulk.

7) When the temperature reaches 35° C. add the premix containing fragrance, phenoxyethanol, diclholorbenzyl alcohol, polysorbate 20 and tocopherol and ensure that the dichlorobenzyl alcohol is fully dissolved and the mix is homogeneous.

8) Mix for 20 minutes and add the Tinopal CBS-X.

9) Add Timica Extra Bright 1500, Timica Silver Sparkle, and mix for 15 minutes to obtain a uniform mix.

10) Add pigment—Pearlescent Pigment Prestige Fire Red—and mix for 30 minutes (or longer until the colour is uniformly distributed throughout the batch).

11) When the temperature reaches 25° C. or below add the ethanol and, after 20 minutes of mixing, follow it up with Silicone DC5225C. Ensure that the vessel is sealed so that Ethanol and Silicone evaporation is minimised.

12) Submit a sample to the laboratory for viscosity test at 20° C. using spindle 5/speed 5: it should be between 18-40,000 cps. The pH should be 6-6.8.

Performance Evaluation

The performance of a composition prepared according to the above example compared to a range of commercially available products marketed as temporary hair colouring preparations was evaluated. The test methods used are given below.

The commercially available compositions tested were L'Oreal Hair Mascara, Jerome Russell Hair Colour Sprays, Smiffy's Hair Colour Spray, Hair Flair, Party Success and got2B Colour Heads (available from Fat Boy Concepts of USA and distributed by Advanced Research Laboratories, Costa Mesa, Calif. 92629) and ‘Hot Head Streak' n Wash-Out Hair Colour’ (available from Hot Head of Glencoe, Ill. 60022, USA)

Test Methods

Instructions for Coating the Hair

1) Apply about 0.75 g of product onto a toothbrush and apply it onto a hair strand. Use 0.75 g of product per hair strand to coat three types of hair darkness (Auburn, Dark and Blonde).

2) Using the toothbrush, brush the product on until it is nearly dry—this should take about 2.5-3 minutes.

3) Using a wide-bristle brush, comb the coated hair while the product is still not totally dry.

4) Allow it to dry and brush again with the wide-bristle brush and compare the colour with the standard sample. The colour coverage should be adequate for a bunch of hair of 15 cm strand length, as supplied by Banbury Postiche (Banbury, OX16 1SR, UK), to coat two thirds of the length of the hair bunch, and leave the hair soft and not stiff (in the manner of a hair-spray or other fixing composition).

Water Resistance Test

1) Staple the coated hair strand (preferably blonde hair) to a sheet of A4 paper.

2) Using a laboratory water bottle squeeze out about 10 ml of water onto the hair strand whilst holding the A4 paper with the hair strand at a 45 degree angle in the sink or over a bench. Allow the water to run over the hair.

3) Observe for any colour coming through with the water as it runs over the hair strand.

4) Allow the hair strand to dry and then lift the hair to see if any discolouration of the paper has taken place, ignoring any solid flakes coming out of hair due to physical handling and concentrating on solubilised colour.

5) There should not be any colour coming out during the gentle squirting from the water bottle or any colour staining of the paper.

Wash-Out Test

1) Choose a blond hair strand coated with colour.

2) Apply warm water and shampoo and rub it with detergent and water for one hair strand over 2 minutes and rinse with warm water for 2 minutes whilst rubbing vigorously.

3) Check that there is no hair colourant left on hair. If there is, rinse again with warm water for 1 minute.

4) The hair must be free from hair colourant.

Results

In contrast to the composition of the invention, only one of the commercially available preparations (got2B Colour Heads) demonstrated any significant resistance to water spray when tested according to the above method. In the case of each of the other commercially available preparations tested, streaking or running of the hair colour was observed to occur when the treated hair was sprayed with water.

Hair coated with the composition according to the invention was found to have a smooth feel and to be easily brushable. The applied composition was removed completely from the hair by simple application of shampoo and warm water.

The product got2B Colour Heads, by contrast, was found to have an undesirable adhesive-type consistency on hair, could not easily be brushed on the hair and was not completely removed on washing with shampoo and warm water.

1. A hair colouring composition in the form of a dispersion comprising
(a) from 0.1% to 25% weight, based on the weight of the composition, of a water-dispersible, sulphonate group-containing polyester polymer,
(b) from 0.5% to 45% weight, based on the weight of the composition, of a pigment, and
(c) from 10% to 95% weight, based on the weight of the composition, of water.

2. A composition according to claim 1 wherein the polyester polymer is a polymer of isophthalic or sulphoisophthalic acid.

3. A composition according to claim 1 wherein the polyester polymer is a copolymer of diglycol/cyclohexanedimethanol/isophthalate/sulphoisophthalate.

4. A composition according to claim 3 wherein the polyester polymer is selected from the group consisting of:
i) a polymer having the properties: approximate molecular weight Mn 7000-8000;
hydroxyl number, <10; acid number, <2; Tg, ° C. (dry polymer), 35-38; inherent viscosity, dL/g, 0.32-0.40;
ii) a polymer having the properties: approximate molecular weight Mn, 6000-7000; hydroxyl number, <10; acid number, <2; Tg, ° C. (dry polymer), 45-48; inherent viscosity, dL/g, 0.26-0.32;
iii) a polymer having the properties: approximate molecular weight Mn 7000-8000;
hydroxyl number, <10; acid number, <2; Tg, ° C. (dry polymer), 51-55; inherent viscosity, dL/g, 0.29-0.37.

5. A composition according to claim 1 wherein the overall non-silicone polymer content is between 0.1% and 10% by weight.

6. A composition according to claim 1 wherein the pigment is selected from the group consisting of an iron oxide colour, carmine red, an indigoid colour, a nitroso dye or a water-insoluble azo dye.

7. A composition according to claim 1 wherein the polyester polymer is present in an amount of from 0.5% to 7.5% by weight, based on the weight of the composition.

8. A composition according to claim 1 wherein the pigment is present in an amount of from 2% to 20% by weight, based on the weight of the composition.

9. A composition according to claim 1 comprising water in an amount of from 30% to 75% by weight, based on the weight of the composition.

10. A composition according to claim 1 additionally comprising an effective amount of at least one cosmetically acceptable ingredient selected from the group consisting of water-miscible organic solvents, plasticizers, spreading agents, de-tangling agents, suspending agents, optical brighteners, preservatives, pH adjusting agents, fragrances and pearlising agents.

11. A composition according to claim 10 comprising a water-miscible organic solvent in an amount of from 5% to 40% by weight.

12. A composition according to claim 1 excluding a hair curl retention additive.

13. A composition according to claim 1 excluding microspheres comprising silica and/or polymethymethacrylate.

14. A composition according to claim 1 comprising one or more surfactants in an amount of 1% or less by weight, based on the weight of the composition.

15. A method for producing a coloured effect in hair which effect can be removed, as and when desired, by washing of the hair, comprising applying to the hair an effective amount of a composition according to claim 1.

16. A product for colouring hair comprising a sachet containing a composition in the form of a dispersion comprising
(a) from 0.1% to 25% weight, based on the weight of the composition, of a water-dispersible, sulphonate group-containing polyester polymer,
(b) from 0.5% to 45% weight, based on the weight of the composition, of a pigment, and
(c) from 10% to 95% weight, based on the weight of the composition, of water, and a dispensing device, in fluid communication with the inside of the sachet, by means of which the composition may be dispensed from the sachet.

17. A product according to claim 16 wherein the composition is dispensed from the sachet by means of dispensing means incorporating a brush or comb and one or more fluid ducts by means of which the composition may be directed to the region of the comb teeth or brush so that it may be dispensed during the use of the brush or comb.

18. A method for reversibly colouring hair, comprising
applying to the hair with a brush or comb a hair colouring composition in the form of a dispersion comprising
from 0.1% to 25% weight, based on the weight of the composition, of a water-dispersible, sulphonate group-containing polyester polymer,
from 0.5% to 45% weight, based on the weight of the composition, of a pigment, and
from 10% to 95% weight, based on the weight of the composition, of water, and thereby transferring the composition to the hair,
whereby the imparted colour is rain resistant but may be removed, as and when desired, by washing of the hair.

19. A method for reversibly colouring hair, comprising
delivering a hair colouring composition in the form of a dispersion comprising from 0.1% to 25% weight, based on the weight of the composition, of a water-dispersible, sulphonate group-containing polyester polymer,
from 0.5% to 45% weight, based on the weight of the composition, of a pigment, and
from 10% to 95% weight, based on the weight of the composition, of water,
from a sachet containing said composition to a brush or comb which is in fluid communication with the inside of said sachet, and
applying the colour to the hair using the brush or comb,
whereby the imparted colour is rain resistant but may be removed, as and when desired, by washing of the hair.
